(19) United States
(12) Patent Application Publication
(10) Pub. No.: US 2019/0038235 A1
(43) Pub. Date: Feb. 7, 2019

(54) DETECTING BLOOD PRESSURE CHANGES

(71) Applicants: Danil Rafikovich Sadriev, Saint-Petersburg (RU); Artur Igorevich Kogdanin, Kazan (RU)

(72) Inventors: Danil Rafikovich Sadriev, Saint-Petersburg (RU); Artur Igorevich Kogdanin, Kazan (RU)

(73) Assignees: Danil Rafikovich Sadriev, Saint-Petersburg (RU); Artur Igorevich Kogdanin, Kazan (RU)

(21) Appl. No.: 16/156,004

(22) Filed: Oct. 10, 2018

Related U.S. Application Data
(63) Continuation-in-part of application No. PCT/RU2016/000211, filed on Apr. 12, 2016.

(51) Int. Cl. A61B 5/00; A61B 5/021

(52) U.S. Cl. CPC A61B 5/7278; A61B 5/7282; A61B 5/6824; A61B 5/742; A61B 5/02125; A61B 5/746

(57) ABSTRACT

Techniques for detecting blood pressure changes are disclosed. A system monitors a user, using two or more sensors fastened to different body parts. Each sensor provides a respective set of measurements. The system computes a phase shift value, between a set of at least two measurements by two respective sensors. The system computes another phase shift value, between another set of at least two measurements by two respective sensors. The system computes a difference between the two phase shift values. The system detects a change in blood pressure, based at least in part on the difference between the two phase shift values.

This application is a continuation-in-part of PCT Application No. PCT/RU2016/000211, filed Apr. 12, 2016, which is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to blood pressure. In particular, the present disclosure relates to detecting blood pressure changes.

### BACKGROUND

In anatomy, blood pressure is a measure of the force applied by blood to the internal walls of blood vessels. For a given location along a blood vessel, factors that contribute to blood pressure at that location include, but are not limited to: the force with which the heart pumps blood through the blood vessel; the diameter of the blood vessel; stiffness of the blood vessel; and elasticity of the blood vessel wall. Blood pressure may vary significantly over time. A blood pulse wave, a.k.a. pulse wave, is a series of measurements over time that represents the rhythmic passage of blood through the blood vessels, caused by the heart's contractions.

Abnormal blood pressure levels and changes in blood pressure are associated with various health risks. Hypertension (i.e., high blood pressure) may indicate an increased risk of aneurysm, heart attacks, cognitive impairment, kidney disease, visual impairment, stroke, and/or other health concerns associated with hypertension. Hypotension (i.e., low blood pressure) may indicate an increased risk of lightheadedness, syncope, visual impairment, fatigue, cognitive impairment, and/or other health concerns associated with hypotension. A rapid change in blood pressure may indicate an increased risk of heart attack, heart disease, stroke, and/or other health concerns associated with a rapid change in blood pressure.

Techniques for measuring blood pressure generally fall into two categories: direct (invasive) techniques and indirect (noninvasive) techniques. Noninvasive techniques rely on indirect indicators relating to hemodynamics and blood pressure. One noninvasive blood pressure measuring technique is the auscultatory method, in which an occluding cuff is inflated around a limb until blood flow is blocked. As the occluding cuff deflates and blood flow resumes, a stethoscope is used to detect sounds from the arterial wall. Another noninvasive blood pressure measuring technique is the oscillometric method, in which an occluding cuff is inflated around a limb and used to measure oscillations in blood pressure flowing through the limb. In some circumstances, the auscultatory method and oscillometric method may be inconvenient and/or impractical. Both methods require inflating the cuff to occlude blood vessels. In addition, the auscultatory method and oscillometric method provide only periodic measurements; they are unable to provide continuous blood pressure monitoring.

Another noninvasive blood pressure measuring technique involves determining the speed of propagation of a pulse wave in the circulatory system. Leading and trailing edges of pulse sequences may be treated as indicators of systolic blood pressure and diastolic blood pressure. See, RF Patent No. RU2118122C1. This technique has relatively low accuracy, because detecting a stable, undistorted pulse wave, by measuring the time difference between two wave fronts of known length, requires fixed connections of sensors to various places on a patient's body.

To measure a pulse wave, a photosensitive transducer may be placed over an area of blood flow. Luminous flux is transformed into an electric signal, which is processed to obtain the pulse wave. See, RF Patent No. RU2199943C2. This technique emphasizes a matrix of sensors for steady pulse detection, rather than changes in blood pressure. In addition, properly orienting a matrix of photosensitive transducers is a complex process, making the technique inconvenient and/or impractical in many circumstances. Improper orientation of a matrix of photosensitive transducers may lead to inaccurate pulse wave measurements.

A relationship has been demonstrated between hemodynamic factors such as pulse wave propagation time and the elasticity of the blood vessel walls. See, Shulgin et. al., Measurement of arterial blood pressure, based on coprocessing of physiological parameters set, Clinical Informatics and Telemedicine 2012 v.8 9^{th} ed. p. 38-44. In addition, phase differences of pulse waves, measured from opposing hands, have been observed as relating to differences in blood vessel hardness on the opposing sides. See, Jin-Kyu Rhee & Sang-Suk Lee, Analysis of Both Hands Two Pulse Waveforms using a Clip-type Pulsimeter Equipped with Magnetic Sensing Hall Device, 18 Journal of Magnetics 183-187 (2013).

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 shows a block diagram that illustrates a system in accordance with an embodiment;

FIG. 2 illustrates an example of a sensor configuration in accordance with an embodiment;

FIG. 3 illustrates an example of pulse waves in accordance with an embodiment;

FIG. 4 illustrates a set of operations for detecting blood pressure changes in accordance with an embodiment;

FIGS. 5A-5E illustrate an example in accordance with an embodiment; and

FIG. 6 shows a block diagram that illustrates a computer system in accordance with one or more embodiments.

### DETAILED DESCRIPTION

In the following description, for the purposes of explanation, numerous specific details are set forth in order to provide a thorough understanding. One or more embodiments may be practiced without these specific details. Features described in one embodiment may be combined with features described in a different embodiment. In some examples, well-known structures and devices are described with reference to a block diagram form in order to avoid unnecessarily obscuring the present invention.

1. GENERAL OVERVIEW

2. ARCHITECTURAL OVERVIEW

3. DETECTING BLOOD PRESSURE CHANGES

4. ILLUSTRATIVE EXAMPLE

5. MISCELLANEOUS; EXTENSIONS

6. HARDWARE OVERVIEW

7. COMPUTER NETWORKS AND CLOUD NETWORKS

### 1. General Overview

One or more embodiments include techniques for detecting blood pressure changes. A system monitors a user, using two or more sensors fastened to different body parts. Each sensor provides a respective set of measurements corresponding to pulse waves. The system computes a phase shift value, between a set of at least two measurements by two respective sensors. The system computes another phase shift value, between another set of at least two measurements by two respective sensors. The system computes a difference between the two phase shift values. A difference in phase shift values over time is considered an indicator of a change in blood pressure. The system detects a change in blood pressure, based at least in part on the difference between the two phase shift values. In an embodiment, the system determines whether the difference between the two phase shift values satisfies a trigger condition that indicates a change in blood pressure. The system may generate a user notification, based on the change in blood pressure.

One or more embodiments described in this Specification and/or recited in the claims may not be included in this General Overview section.

### 2. Architectural Overview

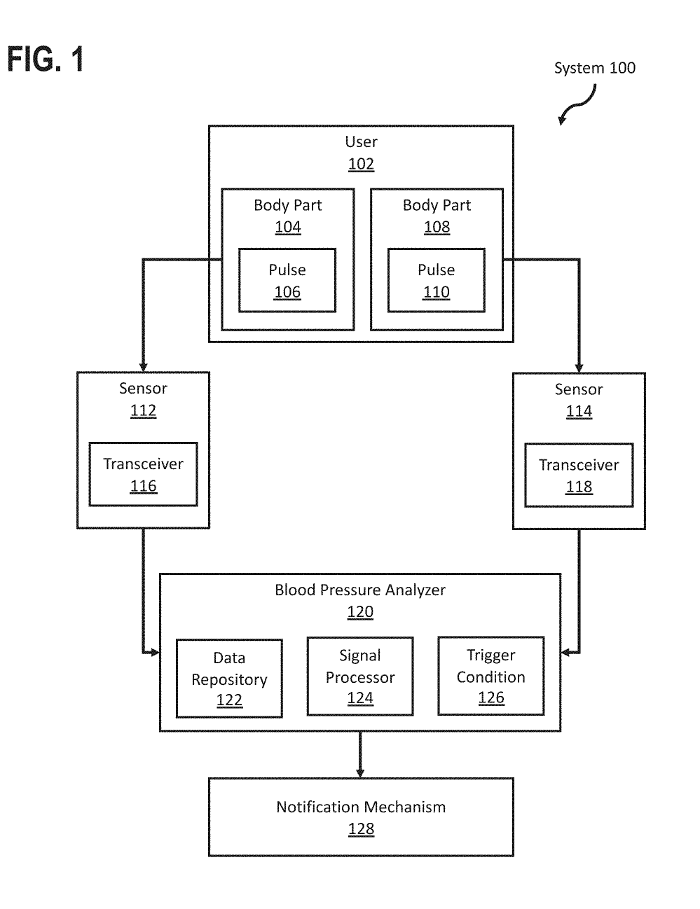

FIG. 1 illustrates a system 100 in accordance with one or more embodiments. As illustrated in FIG. 1, system 100 includes a user 102, two sensors 112, 114, a blood pressure analyzer 120, a notification mechanism 128, and various components thereof. In one or more embodiments, the system 100 may include more or fewer components than the components illustrated in FIG. 1. The components illustrated in FIG. 1 may be local to or remote from each other. One or more of the components illustrated in FIG. 1 may be implemented in software and/or hardware. Each component may be distributed over multiple applications and/or machines. Multiple components may be combined into one application and/or machine. Operations described with respect to one component may instead be performed by another component. Additional embodiments and/or examples relating to computer networks are described below.

In an embodiment, a user 102 is an animal for which blood pressure is to be monitored. For ease of discussion, in examples described herein, the user 102 is assumed to be human. Alternatively, the user 102 may be non-human. The user 102 has two or more body parts 104, 108. Each body part 104, 108 includes at least one location at which a respective pulse 106, 110 is detectable. The body parts 104, 108 may include one or more fingers, wrists, elbows, thighs, calves, ankles, toes, ears, tongue, a torso, or any other body part or combination thereof.

In an embodiment, the sensors 112, 114 are devices configured to take measurements from one or more of the user 102's body parts 104, 108. Specifically, each sensor 112, 114 includes a respective transceiver 116, 118. Each transceiver 116, 118 is configured to take measurements of one or more indicators (e.g., pulse signals) associated with blood pressure at the respective body part 104, 108. In an embodiment, the sensors 112, 114 are configured to obtain measurements corresponding to pulse waves (e.g., plethysmographs and/or any other representations of the pulse waves) for the respective body parts 104, 108. Alternatively or additionally, the sensors 112, 114 may be configured to measure other kinds of blood pressure-related indicators at the respective body part 104, 108.

In an embodiment, a blood pressure analyzer 120 is configured to process data obtained from the sensors 112, 114, to detect blood pressure changes. Specifically, the blood pressure analyzer 120 may include a signal processor 124 configured to obtain signals from the sensors 112, 114 and convert the signals to digital data. The blood pressure analyzer 120 is configured to process the digital data to detect blood pressure changes. Examples of operations for detecting blood pressure changes are described below. One or more components of the blood pressure analyzer 120 may be located in one of the sensors 112, 114. Alternatively or additionally, one or more components of the blood pressure analyzer 120 may be located in a separate device.

In an embodiment, the blood pressure analyzer 120 includes a data repository 122 configured to store data used in detecting blood pressure changes. A data repository 122 may be configured to store data obtained from the sensors 112, 114. Alternatively or additionally, a data repository 122 may be configured to store one or more results of processing the data obtained from the sensors 112, 114. Alternatively or additionally, a data repository 122 may be configured to store baseline data that is used to detect blood pressure changes. Alternatively or additionally, a data repository may be configured to store one or more trigger conditions 126. Alternatively or additionally, a data repository 122 may be configured to store any other kind of data used in detecting blood pressure changes.

In an embodiment, a data repository 122 is any type of storage unit and/or device (e.g., a file system, database, collection of tables, or any other storage mechanism) for storing data. Further, a data repository 122 may include multiple different storage units and/or devices. The multiple different storage units and/or devices may or may not be of the same type or located at the same physical site. Further, a data repository 122 may be implemented or may execute on the same computing system as other components of the system 100. Alternatively or additionally, a data repository 122 may be implemented or executed on a computing system separate from other components of the system 100. A data repository 104 may be communicatively coupled to a blood pressure analyzer 120 and/or another component of the system 100 via a direct connection or via a network.

In an embodiment, a blood pressure analyzer 120 includes one or more trigger conditions 126. A trigger condition 126 may be a condition that, when satisfied, indicates that the user 102's blood pressure has changed. Alternatively or additionally, a trigger condition 126 may be a condition that, when satisfied, indicates that the user 102's blood pressure has changed sufficiently to trigger a user notification. In an embodiment, a trigger condition 126 is designed to detect a change in blood pressure based on one or more of the following observations: (1) an increase in the difference in phase shift values (computed as described below) may indicate a decrease in blood pressure; (2) a rapid increase in the difference in phase shift values may indicate a rapid decrease in blood pressure; (3) a decrease in the difference in phase shift values may indicate an increase in blood pressure; and (4) a rapid decrease in the difference in phase shift values may indicate a rapid increase in blood pressure.

In an embodiment, the sensors 112, 114 and/or different copies of the sensors 112, 114 may be used by multiple users. A trigger condition 126 may be the same for all users. Alternatively or additionally, different trigger conditions may apply to different users. For example, a trigger condition 126 may depend on an attribute of the user 102 such as the user 102's age, sex, height, weight, cholesterol, or any other attribute of the user 102 and/or the user 102's medical history, or combination thereof.

In an embodiment, a trigger condition 126 defines an absolute value. The trigger condition 126 may be satisfied when a value computed by the blood pressure analyzer 120 equals or exceeds the absolute value defined by the trigger condition 126. Alternatively or additionally, a trigger condition 126 may define a percentage value. The trigger condition 126 may be satisfied when a value computed by the blood pressure analyzer 120 deviates from another value by the percentage value defined by the trigger condition 126. Alternatively or additionally, a trigger condition 126 may define a rate of change. The trigger condition 126 may be satisfied when a series of values, computed by the blood pressure analyzer 120 over a period of time, indicates a rate of change that equals or exceeds the rate of change defined by the trigger condition 126. Examples of trigger conditions are discussed in further detail below.

In an embodiment, the system 100 includes a notification mechanism 128. A blood pressure analyzer 120 may use the notification mechanism 128 to present a user notification when a trigger condition 126 is satisfied. The notification mechanism 128 may present many different kinds of user notifications. The notification mechanism 128 may present an audio notification, such as a voice, bell, chime, beep, and/or any other kind of audio notification or combination thereof. Alternatively or additionally, the notification mechanism 128 may present a visual notification, such as a graph (e.g., a graph that represents the user 102's blood pressure changes over time, and/or some other kind of graph), one or more lights (e.g., lights of different colors depending on a state of the user 102's blood pressure, a blinking light, and/or any other kind of light(s)), a text message, an email, and/or any other kind of visual notification or combination thereof. In an embodiment, a user notification indicates a direction of change (i.e., increasing, decreasing, or holding steady) of the user 102's blood pressure. The notification mechanism 128 may present the user notification to the same user 102 whose blood pressure is being monitored. Alternatively or additionally, the notification mechanism 128 may present the user notification to somebody else. In one example, the notification mechanism 128 includes a light fastened to a sensor 114. When a trigger condition 126 is satisfied, the notification mechanism 128 causes the light to start blinking. When another trigger condition 126 is satisfied, the light may cease flashing or begin blinking more rapidly. In another example, the notification mechanism 128 is communicatively coupled to a medical messaging system. When a trigger condition 126 is satisfied, the notification mechanism 128 transmits a message to the user 102's health care provider. In another example, the user 102 designates one or more individuals, such as one or more friends and/or family members, to receive notifications. When a trigger condition 126 is satisfied, the notification mechanism 128 transmits a message to the designated individual(s). Transmitting a notification to somebody other than the user 102 may help reduce a user 102's anxiety associated with blood pressure monitoring.

In an embodiment, one or more components of the system 100 are implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (“PDA”), a wireless receiver and/or transmitter, a base station, a communication management device, a router, a switch, a controller, an access point, and/or a client device.

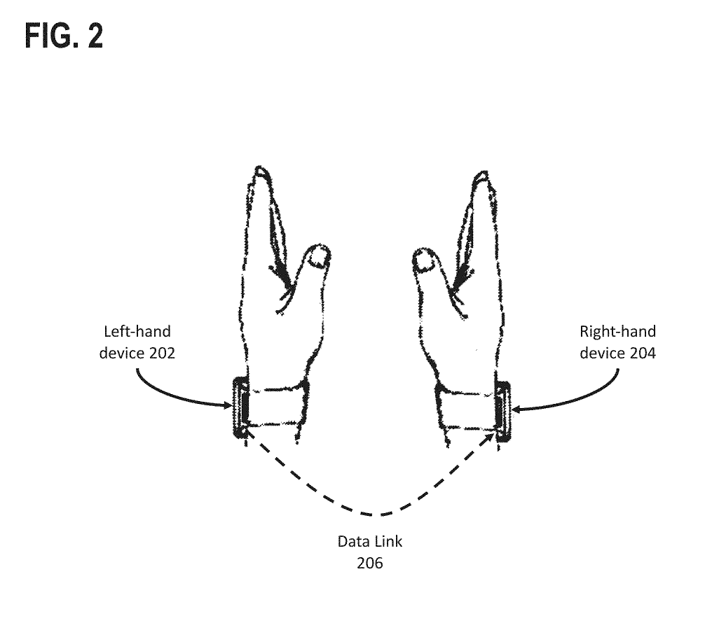

FIG. 2 illustrates an example of a sensor configuration in accordance with an embodiment. Components and/or operations described with respect to FIG. 2 should be understood as one specific example which may not be applicable to certain embodiments. Accordingly, components and/or operations described with respect to FIG. 2 should not be construed as limiting the scope of any of the claims.

As illustrated in FIG. 2, a left-hand device 202 is fastened, using a wristband, to a user's left wrist. A right-hand device 204 is fastened, using a wristband, to a user's right wrist. The left-hand device 202 and right-hand 204 are communicatively coupled via a data link 206. The data link 206 may be a Bluetooth® connection, wi-fi connection, direct cable connection, and/or any other kind of data link 206 or combination thereof. In this example, the left-hand device 202 includes a sensor that is configured to obtain measurements corresponding to pulse waves for the left hand. The right-hand device 204 includes a sensor that is configured to obtain measurements corresponding to pulse waves for the right hand. The right-hand device 204 is configured to transmit data to the left-hand device 202, via the data link 206. The left-hand device 202 includes a blood pressure analyzer that is configured to detect changes in blood pressure, based on data obtained from both sensors. In this example, the blood pressure analyzer computes differences in phase shift values relative to data gathered by the left-hand device 202, i.e., by subtracting right-hand phase shift values from left-hand phase shift values.

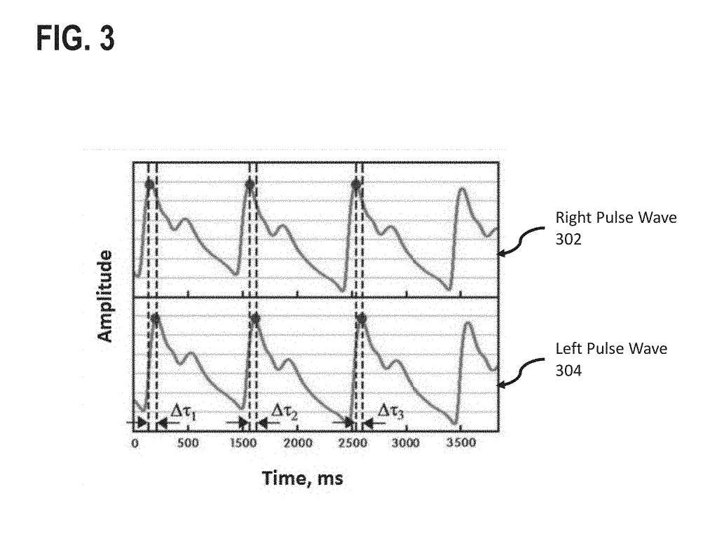

FIG. 3 illustrates an example of pulse waves in accordance with an embodiment. As illustrated in FIG. 3, a right pulse wave 302 represents a pulse wave computed based on data measured using a sensor on a user's right side (e.g., a right wrist, as illustrated in FIG. 2). A left pulse wave 304 represents a pulse wave computed based on data measured using a sensor on a user's left side (e.g., a left wrist, as illustrated in FIG. 2). The right pulse wave 302 and left pulse wave 304 were computed based on concurrent measurements at the two locations. While similar in shape, the two pulse waves do not align precisely. The vertical dashed lines highlight that at various points in time, there are phase differences (e.g., Δτ1, Δτ2, Δτ3) between the right pulse wave 302 and the left pulse wave 304. Phase differences between pulse waves, monitored over time, may be used to detect blood pressure changes. In an embodiment, phase differences between pulse waves are indirect indicators of systolic blood pressure.

### 3. Detecting Blood Pressure Changes

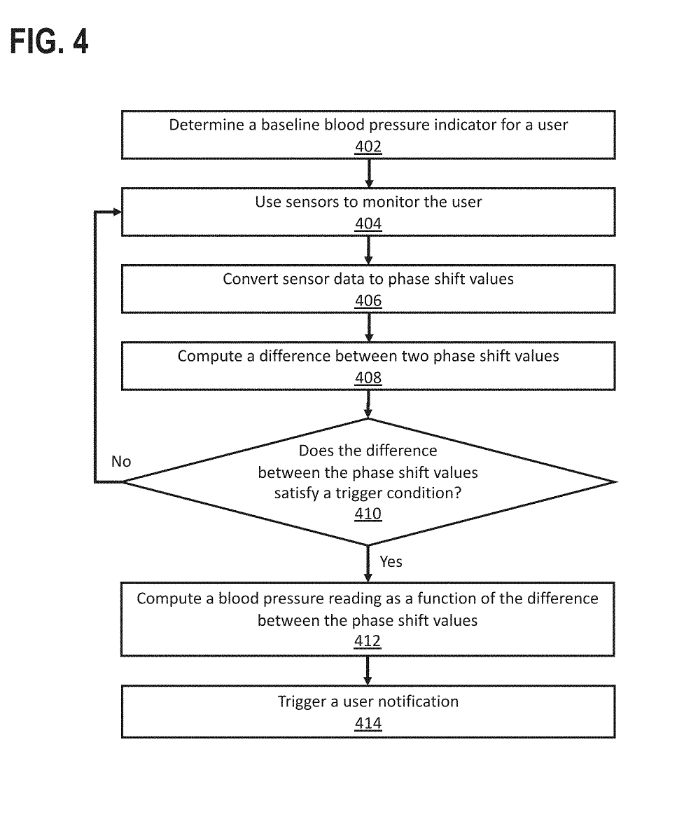

FIG. 4 illustrates an example set of operations for detecting blood pressure changes in accordance with one or more embodiments. One or more operations illustrated in FIG. 4 may be modified, rearranged, or omitted all together. Accordingly, the particular sequence of operations illustrated in FIG. 4 should not be construed as limiting the scope of one or more embodiments.

In an embodiment, operations described herein are based on associations between arterial hardness and both (1) blood pressure and (2) pulse wave propagation time. In addition, operations described herein may be based on differences in arterial hardness between different locations of a body. For example, in humans, arteries may be measurably harder on the left side than on the right side. Accordingly, operations for detecting blood pressure changes may rely on one or more of the following observations: (1) an increase in the difference in phase shift values may indicate a decrease in blood pressure; (2) a rapid increase in the difference in phase shift values may indicate a rapid decrease in blood pressure; (3) a decrease in the difference in phase shift values may indicate an increase in blood pressure; and/or (4) a rapid decrease in the difference in phase shift values may indicate a rapid increase in blood pressure.

In an embodiment, a system determines a baseline blood pressure indicator for a user (Operation 402). A baseline blood pressure indicator may provide a baseline reference point for detecting blood pressure changes. In one example, the baseline blood pressure indicator is a baseline difference between phase shift values. A baseline blood pressure indicator may define an absolute reference value. Alternatively or additionally, a baseline blood pressure indicator may define a range of reference values to be treated as a baseline range.

In an embodiment, the baseline blood pressure indicator is a default value. A blood pressure analyzer may include a factory preset baseline blood pressure indicator. The baseline blood pressure indicator may be the same for multiple users. Alternatively or additionally, different baseline blood pressure indicators may apply to different users. For example, a baseline blood pressure indicator may vary depending on a user's age, sex, height, weight, cholesterol, or any other attribute of the user and/or the user's medical history, or combination thereof. Alternatively or additionally, a system may be configured to determine a baseline blood pressure indicator by monitoring a user over a period of time. In an embodiment, a system is configured to determine a user's baseline blood pressure indicator by using sensors, as described herein, to measure one or more indicators associated with hemodynamics and blood pressure. The system may compute the baseline blood pressure indicator based on a series of measurements taken over time. In one example, a system obtains measurements over a period of minutes, hours, or days. The baseline blood pressure indicator may correspond to a mode, average, or any other function of the data gathered during that time. Alternatively or additionally, a user may input or select a particular baseline blood pressure indicator, via a user interface communicatively coupled with a blood pressure analyzer. Alternatively or additionally, the system may obtain a baseline blood pressure indicator from preexisting medical data associated with the user.

In an embodiment, a system uses sensors to monitor the user (Operation 404). As described above, the sensors are fastened to different body parts on the user. For example, as illustrated in FIG. 2, the sensors may be fastened to the user's left and right wrists. Each sensor is configured to take measurements of one or more indicators relating to blood pressure for the respective body parts. Specifically, each sensor may be configured to obtain measurements corresponding to pulse waves for the respective body parts. In an embodiment, the sensors take measurements concurrently, so that the system is able to compare data obtained by the sensors at approximately the same time. To compare data from two different sensors, the system may require that the measurements be taken sufficiently close in time (e.g., within a range of one millisecond of each other, or some other predefined threshold for the data to be considered sufficiently close in time). The system may discard and/or ignore measurements that are not taken sufficiently close in time.

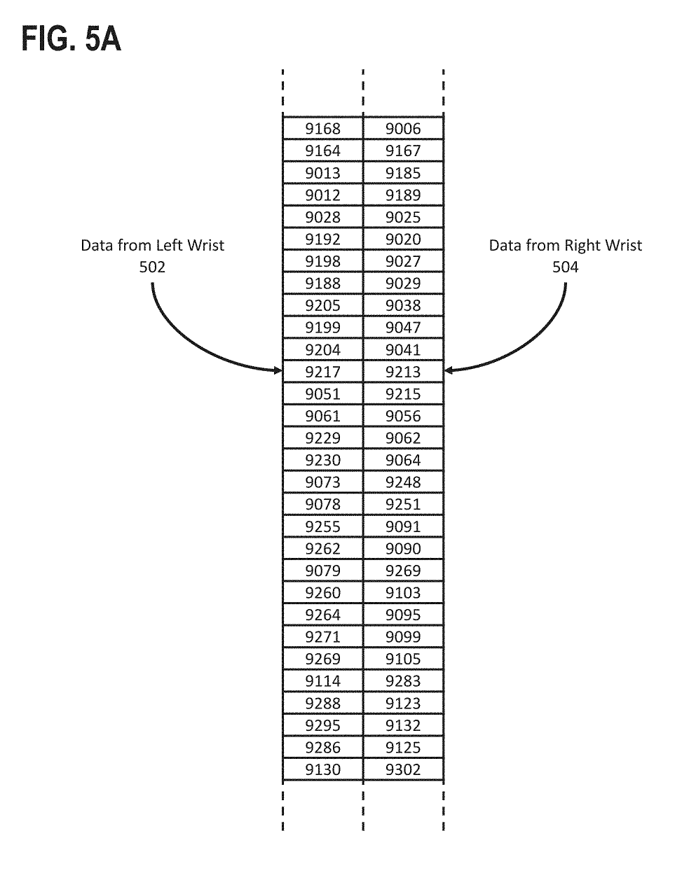

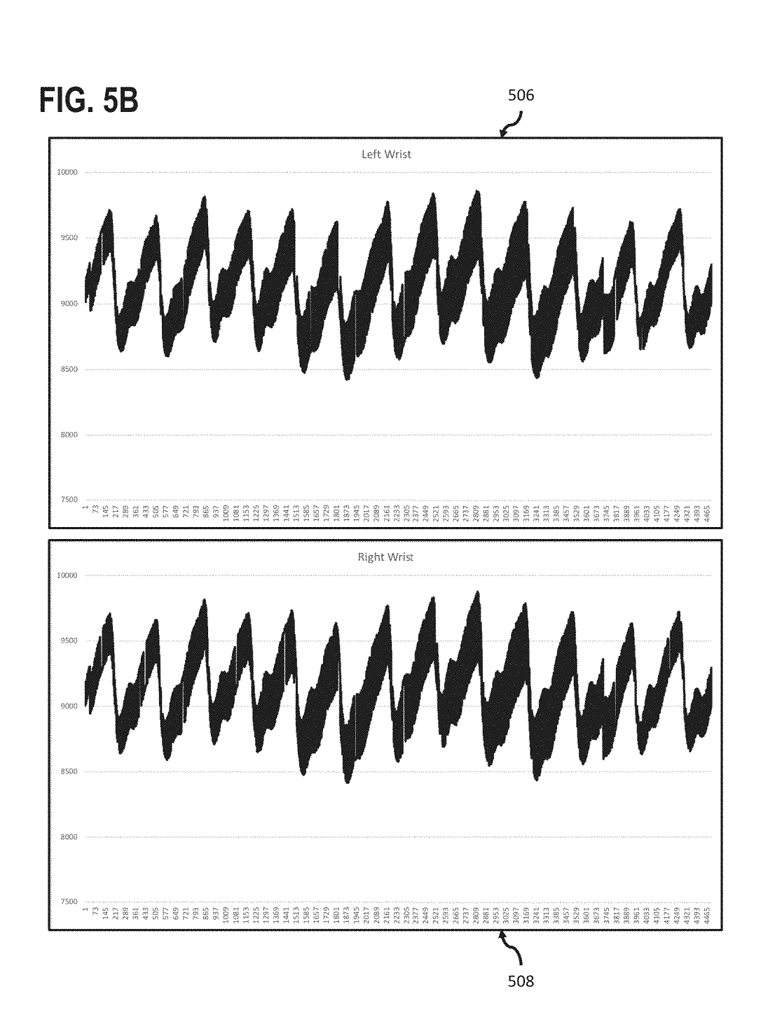

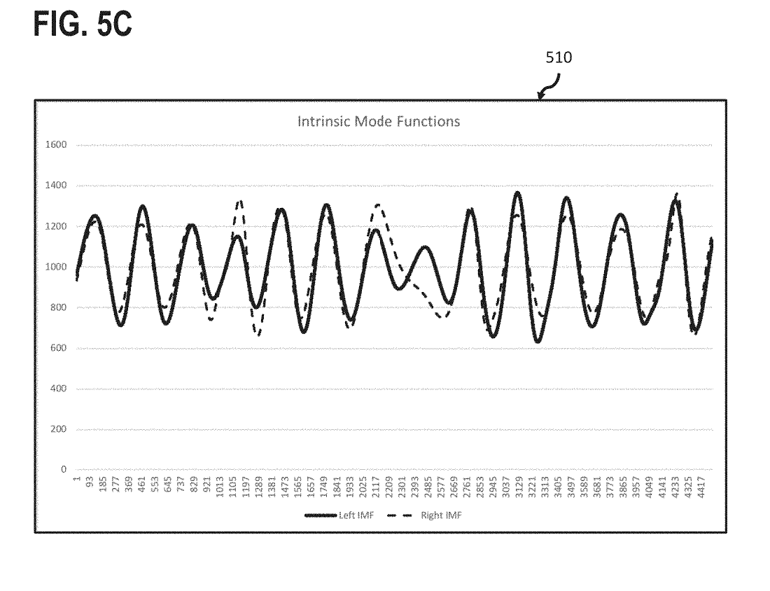

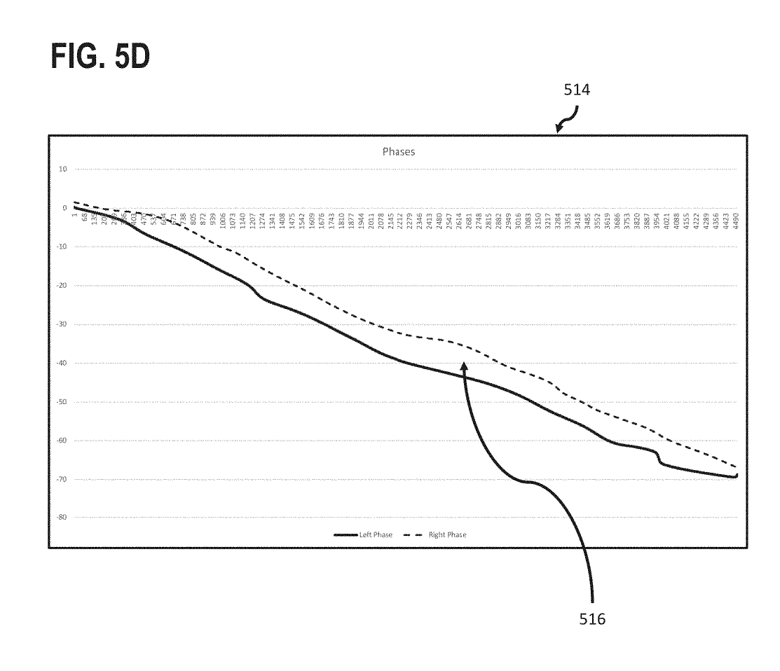

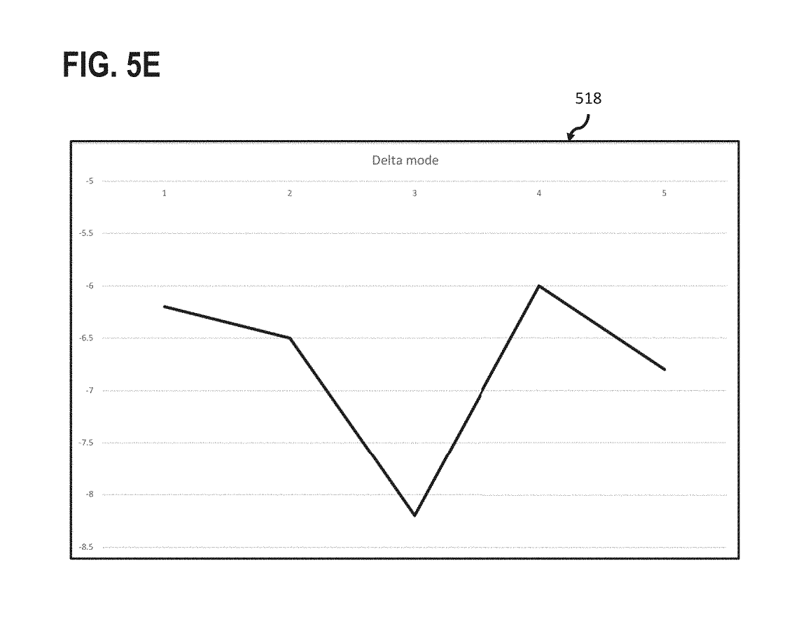

In an embodiment, the system converts sensor data to phase shift values (Operation 406). The system may compute a phase shift value as a function of the difference between two or more pulse waves. Specifically, a phase shift value may be a function of the amount of time (e.g., nanoseconds, milliseconds, or any other measure of time) between a particular point in one pulse wave (e.g., a leading edge, a trailing edge, a peak, or any other discernible point) and a corresponding point in another pulse wave. For example, as illustrated in FIG. 3, a phase shift value may be a function of (a) the time when a right-hand sensor reads data corresponding to the peak of a right pulse wave 302 and (b) the time when a left-hand sensor reads data corresponding to the peak of a left pulse wave 304. Alternatively or additionally, a system may compute a phase shift value as a function of multiple phase shift values. The system may compute a mode, average, maximum, minimum, or any other function of multiple phase shift values, or combination thereof. An example of computing phase shift values is discussed below with reference to FIGS. 5A-5E.

In an embodiment, a system computes a difference between two phase shift values (Operation 408). The difference between the two phase shift values indicates a change in phase shift values over time. One phase shift value is a historical phase shift value (e.g., a phase shift value computed for a preceding point in time, a mode of phase shift values computed for a preceding period of time, or any other historical phase shift value, or combination thereof). The other phase shift value is a more recent phase shift value (e.g., a phase shift value computed for a current set of sensor data, a mode of phase shift values computed for a recent period of time, or any other recent phase shift value, or combination thereof).

In one example, a system computes a mode of phase shift values over a period of time. In addition, the system computes a phase shift value based on a more recent set of sensor data. In this example, the system computes a difference in phase shift values as the difference between the historical mode and the more recent phase shift value. In another example, the system computes a mode of phase shift values over a period of time. In addition, the system computes another mode of phases shift values over a more recent period of time. In this example, the system computes a difference in phase shift values as the difference between the historical mode and the more recent mode. The system may compute a difference between two phase shift values in many different ways. In an embodiment, a system uses a baseline blood pressure indicator as a historical phase shift value.

In an embodiment, a system determines whether the difference between the phase shift values satisfies a trigger condition (Operation 410). As noted above, a trigger condition may be a condition that, when satisfied, indicates that the user's blood pressure has changed. Alternatively or additionally, a trigger condition may be a condition that, when satisfied, indicates that the user's blood pressure has changed sufficiently to trigger a user notification.

In one example, the trigger condition defines an absolute value corresponding to a threshold difference in phase shift values. The trigger condition is satisfied when a difference in phase shift values exceeds the threshold difference defined by the trigger condition. In another example, the trigger condition defines an absolute value corresponding to a threshold change in the difference between phase shift values. The trigger condition is satisfied when a recent phase shift value deviates from a historical phase shift value by at least the threshold amount. In another example, a trigger condition defines a percentage value. The trigger condition is satisfied when a recent phase shift value falls above or below a historical phase shift value by the percentage defined by trigger condition. In another example, a trigger condition defines a rate of change. The trigger condition is satisfied when a recent phase shift value deviates from a historical phase shift value by a threshold amount within a predefined amount of time. Many different kinds of trigger conditions, or combinations thereof, may be used.

In an embodiment, a system requires that a trigger condition be satisfied more than once, to account for noise in the data such as natural fluctuations in phase shift values and/or erroneous sensor readings. In one example, a current phase shift value exceeds a historical phase shift value by a threshold amount defined by a trigger condition. The system continues monitoring the user. If a subsequent phase shift value also exceeds the historical phase shift value by the threshold amount, the trigger condition is satisfied. A system may require that a trigger condition be satisfied more than twice. Alternatively or additionally, a system may require that the trigger condition be satisfied two or more times within a predefined time period.

In an embodiment, the difference between the phase shift values does not satisfy the trigger condition, the system continues using the sensors to monitor the user (Operation 404). As the system continues to monitor the user, the system may incorporate recent sensor data into one or more historical phase shift values. In one example, a phase shift value that was previously treated as a current phase shift value is incorporated into a mode of historical phase shift values. In this example, measurements that are subsequently obtained from the sensors are used to compute a more recent phase shift value, which is compared to the updated mode. In an embodiment, if the difference between the phase shift values satisfies the trigger condition, the system may also continue monitoring the user, while performing operations described below.

In an embodiment, if the difference between the phase shift values satisfies the trigger condition, the system computes a blood pressure reading as a function of the difference between the phase shift values (Operation 412). The blood pressure reading may correspond to a systolic blood pressure and/or diastolic blood pressure. The blood pressure reading may be measured in millimeters of mercury (mmHg) and/or any other unit(s) suitable for quantifying blood pressure. In an embodiment, the system computes the blood pressure reading before evaluating a trigger condition. The system may evaluate a trigger condition as a function of the blood pressure reading. Alternatively, the system may not compute a blood pressure reading. In an embodiment, phase shift values are sufficient to detect changes in blood pressure without ever computing a blood pressure reading. The system may compute a blood pressure reading for informational purposes only.

In an embodiment, the system uses deep learning techniques to compute a blood pressure reading. The deep learning techniques may include, but are not limited to, application of a neural network to user data. The neural network (or another kind of deep learning engine) may receive data associated with one or more users and, based on the data, learn how to compute a blood pressure reading. The user data may include differences between phase shift values, other physiological parameters, known blood pressure values, and/or any other kind of data associated with hemodynamics and/or blood pressure. For a particular user, the system may supply data obtained as described herein. Responsive to the user data, the neural network may compute a blood pressure value.

In an embodiment, a system triggers a user notification (Operation 414). The system may trigger a user notification when a single trigger condition is satisfied. Alternatively, the system may require that multiple trigger conditions are satisfied before triggering a user notification. In one example, the system stores two or more trigger conditions. One trigger condition merely indicates that a user's blood pressure has changed. Another trigger condition indicates that a user's blood pressure has changed enough (i.e., by a sufficiently large amount and/or at a sufficiently fast rate) to trigger a user notification. As discussed above, a user notification may take many forms.

### 4. Illustrative Example

A detailed example is described below for purposes of clarity. Components and/or operations described below should be understood as one specific example which may not be applicable to certain embodiments. Accordingly, components and/or operations described below should not be construed as limiting the scope of any of the claims.

FIGS. 5A-5E illustrate an example in accordance with an embodiment. Specifically, FIG. 5A illustrates a set of data gathered from sensors, in accordance with an embodiment. In the illustration of FIG. 5A, the data is organized into two columns, corresponding to data from a user's left wrist 502 and data from a user's right wrist 504. Each row corresponds to readings taken from the left and right sides, respectively, at approximately the same time. Each data item is a measure of the amount of light reflected from the skin surface at the location of a blood vessel, in units of analog-to-digital converter (ADC) counts/lux. The data illustrated in FIG. 5A is only a small subset of data. In practice, each sensor may measure hundreds, thousands, or millions of values over time. FIG. 5B illustrates the sensor data in graph form, including a graph 506 of the data from the user's left wrist 502 and a graph 508 of the data from the user's right wrist 504.

In an embodiment, a system computes intrinsic mode functions based on the sensor data. Specifically, the system may use Empirical Mode Decomposition (EMD), or any other function(s) or combination thereof, to obtain intrinsic mode functions for the data from each sensor. The system computes/detects intrinsic mode functions that include pulse wave signals. FIG. 5C illustrates overlaid graphs 510 of intrinsic mode functions that include pulse wave signals based on sensor data. In the overlaid graphs 510, the intrinsic mode function based on the data from the user's left wrist 502 is represented as a solid line. The intrinsic mode function based on the data from the user's right wrist 504 is represented as a dashed line.

In an embodiment, a system converts the intrinsic mode functions to phases. Specifically, the system may apply a Hilbert transformation, or any other function(s) or combination thereof, to obtain phases of pulse wave signals from the intrinsic mode functions corresponding to each wrist. FIG. 5D illustrates overlaid graphs 514 of phases of the intrinsic mode functions illustrated in FIG. 5C. In the overlaid graphs 510, the phase based on the data from the user's left wrist 502 is represented as a solid line. The phase based on the data from the user's right wrist 504 is represented as a dashed line. At any given point along the x-axis, the space 516 between the two graphs along the y-axis corresponds to the difference (a.k.a. delta) between the two phase values. In an embodiment, to compute the phase, the system uses only those sequences of data that include a threshold number of heartbeats (e.g., two or three heartbeats). Alternatively or additionally, the system may use only those sequences of data where the pulse remains in a predefined range (e.g., sixty (60) to one hundred twenty (120) beats per minute, or any other predefined range). Alternatively or additionally, the system may use only those sequences of data where the pulse does not vary by more than a predefined percentage (e.g., one percent (1%), or any other predefined percentage). One or more of the aforementioned techniques may help filter out possible noise in the data, such as reading errors caused by motion, artifacts, etc.

In an embodiment, a system computes one or more modes of the phase values represented in FIG. 5C. FIG. 5E illustrates a graph 518 of modes of phase shift values. In this example, the system has computed modes for phase shift values in increments of nine hundred (900) delta measurements. The lines of the graph 518 connect the modes for each increment. Alternatively, a system may calculate modes for smaller subsets of delta measurements, such as for increments of every one hundred (100) delta measurements, fifty (50) delta measurements, ten (10) delta measurements, one (1) delta measurement, etc. Modes calculated in this manner may be treated as phase shift values for the purpose of detecting a change in blood pressure.

In an embodiment, detecting blood pressure changes as described herein allows for efficient, reliable, nonintrusive blood pressure monitoring. A system as described herein may avoid the inconvenience of inflating an occluding cuff, while allowing for continuous monitoring rather than only period measurements. Detecting a direction and magnitude of change may be sufficient for a user to make health-related decisions, without requiring the system to compute a precise blood pressure reading. A system as described herein may be suitable for situations where other blood pressure monitoring and/or measurement techniques are not suitable, such as while driving. An ability to monitor blood pressure changes in a broader range of circumstances may allow a user to make health decisions (e.g., whether to take a pill to regulate blood pressure) in a more timely manner than if the user had to wait to have access to blood pressure monitoring and/or measurement tools.

### 5. Miscellaneous; Extensions

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

### 6. Hardware Overview

According to one embodiment, the techniques described herein are implemented by one or more special-purpose computing devices (i.e., computing devices specially configured to perform certain functionality). The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

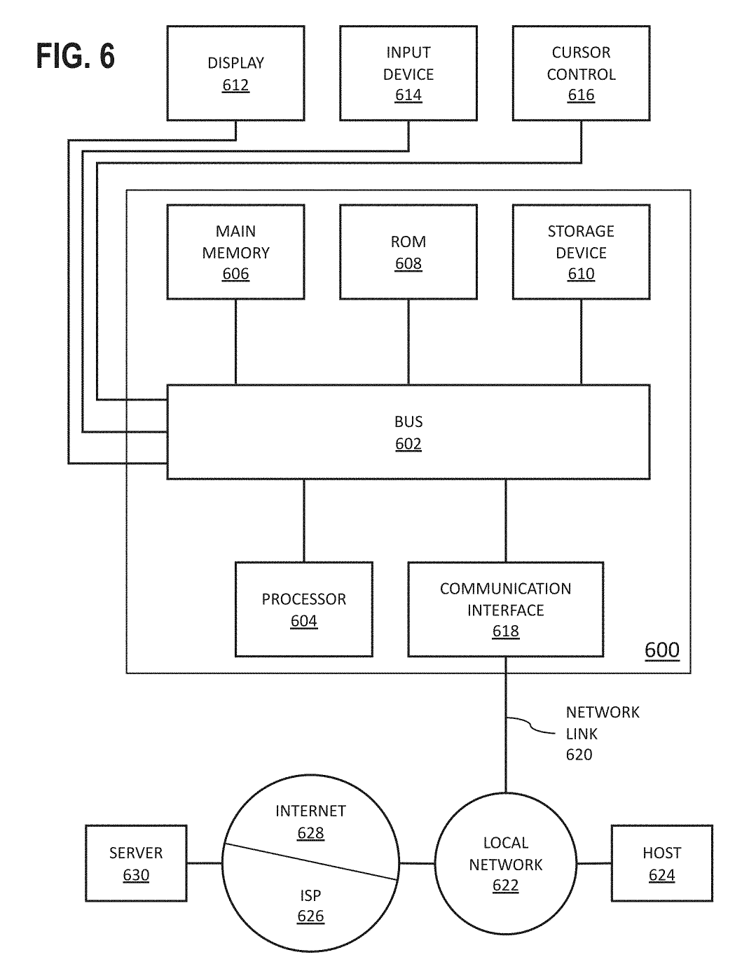

For example, FIG. 6 is a block diagram that illustrates a computer system 600 upon which an embodiment of the invention may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read only memory (ROM) 608 or other static storage device coupled to bus 602 for storing static information and instructions for processor 604. A storage device 610, such as a magnetic disk or optical disk, is provided and coupled to bus 602 for storing information and instructions.

Computer system 600 may be coupled via bus 602 to a display 612, such as a liquid crystal display (LCD), plasma display, electronic ink display, cathode ray tube (CRT) monitor, or any other kind of device for displaying information to a computer user. An input device 614, including alphanumeric and other keys, may be coupled to bus 602 for communicating information and command selections to processor 604. Alternatively or in addition, the computer system 600 may receive user input via a cursor control 616, such as a mouse, a trackball, a trackpad, a touchscreen, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane. The display 612 may be configured to receive user input via one or more pressure-sensitive sensors, multi-touch sensors, and/or gesture sensors. Alternatively or in addition, the computer system 600 may receive user input via a microphone, video camera, and/or some other kind of user input device (not shown).

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a programmable read-only memory (PROM), and erasable PROM (EPROM), a FLASH-EPROM, non-volatile random-access memory (NVRAM), any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a network, via a network interface controller (NIC), such as an Ethernet controller or Wi-Fi controller. A NIC local to computer system 600 can receive the data from the network and place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 7. Computer Networks and Cloud Networks

In one or more embodiments, a computer network provides connectivity among a set of nodes running software that utilizes techniques as described herein. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be any physical resource that provides compute power to perform a task, such as one that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis. Network resources assigned to each request and/or client may be scaled up or down based on, for example, (a) the computing services requested by a particular client, (b) the aggregated computing services requested by a particular tenant, and/or (c) the aggregated computing services requested of the computer network. Such a computer network may be referred to as a “cloud network.”

In an embodiment, a service provider provides a cloud network to one or more end users. Various service models may be implemented by the cloud network, including but not limited to Software-as-a-Service (SaaS), Platform-as-a-Service (PaaS), and Infrastructure-as-a-Service (IaaS). In SaaS, a service provider provides end users the capability to use the service provider's applications, which are executing on the network resources. In PaaS, the service provider provides end users the capability to deploy custom applications onto the network resources. The custom applications may be created using programming languages, libraries, services, and tools supported by the service provider. In IaaS, the service provider provides end users the capability to provision processing, storage, networks, and other fundamental computing resources provided by the network resources. Any applications, including an operating system, may be deployed on the network resources.

In an embodiment, various deployment models may be implemented by a computer network, including but not limited to a private cloud, a public cloud, and a hybrid cloud. In a private cloud, network resources are provisioned for exclusive use by a particular group of one or more entities (the term “entity” as used herein refers to a corporation, organization, person, or other entity). The network resources may be local to and/or remote from the premises of the particular group of entities. In a public cloud, cloud resources are provisioned for multiple entities that are independent from each other (also referred to as “tenants” or “customers”). The computer network and the network resources thereof are accessed by clients corresponding to different tenants. Such a computer network may be referred to as a “multi-tenant computer network.” Several tenants may use a same particular network resource at different times and/or at the same time. The network resources may be local to and/or remote from the premises of the tenants. In a hybrid cloud, a computer network comprises a private cloud and a public cloud. An interface between the private cloud and the public cloud allows for data and application portability. Data stored at the private cloud and data stored at the public cloud may be exchanged through the interface. Applications implemented at the private cloud and applications implemented at the public cloud may have dependencies on each other. A call from an application at the private cloud to an application at the public cloud (and vice versa) may be executed through the interface.

In an embodiment, tenants of a multi-tenant computer network are independent of each other. For example, one tenant (through operation, tenant-specific practices, employees, and/or identification to the external world) may be separate from another tenant. Different tenants may demand different network requirements for the computer network. Examples of network requirements include processing speed, amount of data storage, security requirements, performance requirements, throughput requirements, latency requirements, resiliency requirements, Quality of Service (QoS) requirements, tenant isolation, and/or consistency. The same computer network may need to implement different network requirements demanded by different tenants.

In one or more embodiments, in a multi-tenant computer network, tenant isolation is implemented to ensure that the applications and/or data of different tenants are not shared with each other. Various tenant isolation approaches may be used.

In an embodiment, each tenant is associated with a tenant ID. Each network resource of the multi-tenant computer network is tagged with a tenant ID. A tenant is permitted access to a particular network resource only if the tenant and the particular network resources are associated with a same tenant ID.

In an embodiment, each tenant is associated with a tenant ID. Each application, implemented by the computer network, is tagged with a tenant ID. Additionally or alternatively, each data structure and/or dataset, stored by the computer network, is tagged with a tenant ID. A tenant is permitted access to a particular application, data structure, and/or dataset only if the tenant and the particular application, data structure, and/or dataset are associated with a same tenant ID.

As an example, each database implemented by a multi-tenant computer network may be tagged with a tenant ID. Only a tenant associated with the corresponding tenant ID may access data of a particular database. As another example, each entry in a database implemented by a multi-tenant computer network may be tagged with a tenant ID. Only a tenant associated with the corresponding tenant ID may access data of a particular entry. However, the database may be shared by multiple tenants.

In an embodiment, a subscription list indicates which tenants have authorization to access which applications. For each application, a list of tenant IDs of tenants authorized to access the application is stored. A tenant is permitted access to a particular application only if the tenant ID of the tenant is included in the subscription list corresponding to the particular application.

In an embodiment, network resources (such as digital devices, virtual machines, application instances, and threads) corresponding to different tenants are isolated to tenant-specific overlay networks maintained by the multi-tenant computer network. As an example, packets from any source device in a tenant overlay network may only be transmitted to other devices within the same tenant overlay network. Encapsulation tunnels are used to prohibit any transmissions from a source device on a tenant overlay network to devices in other tenant overlay networks. Specifically, the packets, received from the source device, are encapsulated within an outer packet. The outer packet is transmitted from a first encapsulation tunnel endpoint (in communication with the source device in the tenant overlay network) to a second encapsulation tunnel endpoint (in communication with the destination device in the tenant overlay network). The second encapsulation tunnel endpoint decapsulates the outer packet to obtain the original packet transmitted by the source device. The original packet is transmitted from the second encapsulation tunnel endpoint to the destination device in the same particular overlay network.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A system comprising:
at least one device including a hardware processor;
the system being configured to perform operations comprising:
monitoring a user using each of a plurality of sensors;
computing a first phase shift value between a first set of at least two measurements by two respective sensors from the plurality of sensors;
computing a second phase shift value between a second set of at least two measurements by the two respective sensors from the plurality of sensors;
computing a difference between the first phase shift value and the second phase shift value; and
detecting a change in blood pressure, based at least in part on the difference between the first phase shift value and the second phase shift value.

2. The system of claim 1, wherein detecting the change in blood pressure comprises determining that the difference between the first phase shift value and the second phase shift value satisfies a first trigger condition.

3. The system of claim 2, wherein detecting the change in blood pressure is further based on:
computing a third phase shift value between a third set of at least two measurements by the two respective sensors from the plurality of sensors;
computing a difference between the first phase shift value and the third phase shift value; and
determining that the difference between the first phase shift value and the third phase shift value satisfies the first trigger condition.

4. The system of claim 2, the operations further comprising:
determining that the difference between the first phase shift value and the second phase shift value satisfies a second trigger condition;
responsive to determining that the difference between the first phase shift value and the second phase shift value satisfies the second trigger condition, triggering a user notification corresponding to a rapid change in blood pressure.

5. The system of claim 2, wherein the first trigger condition is based on one or more attributes of the user.

6. The system of claim 2, wherein the first trigger condition defines an absolute value.

7. The system of claim 2, wherein the first trigger condition defines a percentage value.

8. The system of claim 2, wherein the first trigger condition defines a rate of change.

9. The system of claim 1, the operations further comprising:
determining a baseline blood pressure indicator for the user, wherein detecting the change in blood pressure comprises computing a deviation from the baseline blood pressure indicator.

10. The system of claim 1, the operations further comprising:
responsive to detecting the change in blood pressure: generating a visual notification of the change in blood pressure.

11. The system of claim 10, wherein the visual notification comprises a notification of a direction of the change in blood pressure.

12. The system of claim 10, wherein the visual notification comprises a change in blink frequency of a light.

13. The system of claim 1, the operations further comprising:
computing a blood pressure reading as a function of the difference between the first phase shift value and the second phase shift value.

14. The system of claim 1, the operations further comprising:
selecting the first phase shift value based on a mode of measurements taken over a period of time.

15. The system of claim 1, wherein the plurality of sensors comprises a first sensor fastened to a first appendage of the user and a second sensor fastened to a second appendage of the user.

16. The system of claim 1, wherein the first appendage is the user's left wrist and the second appendage is the user's right wrist.

17. The system of claim 1, wherein the plurality of sensors are configured to obtain a plurality of photoplethysmograms.

18. The system of claim 1, wherein detecting the change in blood pressure is performed by a particular sensor from the plurality of sensors.

19. A non-transitory computer readable medium comprising instructions which, when executed by one or more hardware processors, cause performance of operations comprising:
monitoring a user using each of a plurality of sensors;
computing a first phase shift value between a first set of at least two measurements by two respective sensors from the plurality of sensors;
computing a second phase shift value between a second set of at least two measurements by the two respective sensors from the plurality of sensors;
computing a difference between the first phase shift value and the second phase shift value; and
detecting a change in blood pressure, based at least in part on the difference between the first phase shift value and the second phase shift value.

20. A method comprising:
monitoring a user using each of a plurality of sensors;
computing a first phase shift value between a first set of at least two measurements by two respective sensors from the plurality of sensors;
computing a second phase shift value between a second set of at least two measurements by the two respective sensors from the plurality of sensors;
computing a difference between the first phase shift value and the second phase shift value; and
detecting a change in blood pressure, based at least in part on the difference between the first phase shift value and the second phase shift value,
wherein the method is performed by at least one device comprising a hardware processor.
